(19) United States
(12) Patent Application Publication
(10) Pub. No.: US 2008/0166261 A1
(43) Pub. Date: Jul. 10, 2008

(54) System and Method for Sterilizing and/or Disinfecting Instruments Used on Human Beings

(76) Inventors: Michael O'Keefe, Medford, NJ (US); Gail O'Keefe, Medford, NJ (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/971,418

(22) Filed: Jan. 9, 2008

Related U.S. Application Data
(60) Provisional application No. 60/884,240, filed on Jan. 10, 2007.

(51) Int. Cl. A61L 2/07; G06Q 90/00

(52) U.S. Cl. 422/26; 422/297; 705/1

(57) ABSTRACT

A method for providing services to a customer of a nail salon is disclosed. The method includes placing at least one instrument for use in performing a manicure and/or pedicure into a sterilization pouch. The sterilization pouch is sealed and processed in an autoclave in order to sterilize the instrument(s) within the pouch. The pouch is then unsealed in the presence of the customer, prior to using the instrument(s) to perform the manicure and/or pedicure. Optionally, the customer may be explicitly informed that the instrument(s) is sterile, verbally and/or by a writing.

### BACKGROUND OF THE INVENTION

This invention relates to sterilizing and/or disinfecting instruments used on human beings, and more particularly to a system and method for sterilizing and/or disinfecting instruments used in nail salons by means of an autoclave.

In the medical field, it is standard practice to sterilize reusable instruments between uses on different patients. Sterilization effectively eliminates all transmissible agents, such as bacteria and viruses, from the instruments. This prevents the transmission of blood-borne pathogens from the instruments to the patient. The most common blood-borne infections in the United States are hepatitis B, hepatitis C and human immunodeficiency virus (HIV). Thus, to prevent the spread of these potentially fatal diseases, sterilization of reusable medical instruments which come into contact with blood, fluids containing blood and/or bodily tissue, is essential.

A widely-used method to sterilize medical instruments is by means of an autoclave. Autoclaves achieve sterilization through heated steam. Commonly, autoclaves use steam heated to about 121° C. at a pressure of 103 kPA for a period of at least 15 minutes. Prior to processing instruments in an autoclave, any biological debris on the instruments should be removed. This may be achieved via a variety of methods, including physical scrubbing with detergent and water, an ultrasound bath and/or pulsed air. An ultrasonic bath operates on the principle of cavitation, in which sound waves are pulsed through a mixture of detergent and water, thereby causing tiny bubbles to form on the surface of the objects to be cleaned. The bubbles then implode, pulling organic debris off of the objects.

There are various indicators available to ensure that an autoclave process has performed effectively. One common indicator is a chemical indicator, which changes color once the conditions for sterilization inside the autoclave have been met. Often, reusable instruments are placed inside of sterilization pouches featuring such a color indicator. Sterilization pouches may be used in an autoclave, permitting the contents of the pouches to be sterilized. An example of such a pouch having a color-coded processing indicator is described in U.S. Pat. No. 5,344,017 (Wittrock) whose disclosure is incorporated herein by reference.

Sterilization pouches with processing indicators are commonly used, among other places, in dental offices. After dental instruments have been used on a patient, they are commonly: (1) cleansed in an ultrasonic bath; (2) rinsed; and (3) sealed inside a sterilization pouch, which is processed in its entirety in an autoclave. Once the autoclaving process is complete, the indicator on the sterilization pouch informs the autoclave operator that the instruments within the pouch have been properly sterilized. Through this method of sterilization, dentists prevent the spread of infection to their patients.

Such a method of sterilization is currently employed in dental, medical and veterinary offices and hospitals. However, the problem of transmission of dangerous blood-borne pathogens by means of reusable instruments is not limited to the context of medical treatment. Reuse of instruments in nail salons presents a dangerous risk of spreading infection. In a nail salon, various instruments, e.g., nail files, cuticle sticks, nail clippers and cuticle nippers are used and then reused on different customers. Such instruments contact bodily tissue and sometimes bodily fluids, and therefore carry the serious risk of transmitting infection from one customer to another. Despite this risk, nail salons do not routinely sterilize their reusable instruments. Some nail salons may employ some means to treat their reusable instruments, such as liquid disinfectants or ultraviolet radiation, but such methods are not always reliable.

Lack of, or inadequate sterilization of reusable instruments in a nail salon is dangerous for customers and bad for the salon's business. Even experiencing a minor infection may dissuade some customers from returning to a nail salon for a manicure or pedicure. Additionally, liability for transmission of a dangerous disease could potentially devastate a nail salon's business. Symptoms of some dangerous blood-borne diseases do not manifest themselves for years after the disease-causing pathogen first enters the body. For example, a person could be infected with hepatitis C for up to 30 years without feeling symptoms of the liver-destroying disease. Thus, unsuspecting customers of nail salons are at risk of contracting diseases that they may not even know that they have until years later. Such unfortunate persons will be unaware of the cause of their disease.

Therefore, there exists a need for a system and method for sterilization of reusable instruments in a nail salon. This need is felt both by customers, who are at risk for contracting diseases in nail salons, as well as the salons themselves, who would benefit from the tremendous business advantage inherent in being known to use sterilized instruments. To this end, there is a further need for the system or method to make apparent to customers, the fact that the instruments are sterilized.

### BRIEF SUMMARY OF THE INVENTION

The invention disclosed and claimed herein addresses the needs presented above. In one embodiment, a system for sterilizing and/or disinfecting instruments to be used for manicures and/or pedicures is disclosed. The system comprises an autoclave and a sterilization pouch containing at least one manicure and/or pedicure instrument. In another embodiment of the invention, a method of sterilizing and/or disinfecting instruments to be used for manicures and/or pedicures is disclosed. This method involves placing at least one of the instruments into a sterilization pouch, sealing the pouch and processing the pouch in an autoclave in order to sterilize and/or disinfect the instrument(s). Yet another embodiment of the invention is a method for providing services to a customer of a nail salon. This method involves placing at least one instrument used for manicures and/or pedicures on a customer into a sterilization pouch, sealing the pouch, processing the pouch in an autoclave in order to sterilize and/or disinfect the instrument(s) and unsealing the pouch in the customer's presence before using the instrument to perform a manicure and/or pedicure on the customer. Still another embodiment of the invention is a method for providing services to a customer of a nail salon, involving unsealing, in the presence of the customer, a package containing at least one disposable instrument to be used for performing a manicure and/or pedicure on the customer.

### BRIEF DESCRIPTION OF SEVERAL VIEWS OF THE DRAWINGS

The invention will be described in conjunction with the following drawings in which like reference numerals designate like elements and wherein:

FIG. 1 is a block diagram illustrating a method for providing services for a customer in a nail salon, where the customer is to receive a manicure and/or pedicure with a reusable instrument(s).

FIG. 2 is a block diagram illustrating a method of sterilizing an instrument(s) to be used for a manicure and/or pedicure.

FIG. 3 is a block diagram illustrating a method for providing services for a customer in a nail salon, where the customer is to receive a manicure and/or pedicure with a disposable instrument(s).

### DETAILED DESCRIPTION OF THE INVENTION

One embodiment of the present invention (not shown) includes a system to be used in a nail salon for sterilization of reusable instruments used to perform a manicure and/or pedicure on a customer. The system comprises an autoclave and a sterilization pouch containing at least one reusable instrument. The system may be implemented for a method of sterilization of reusable instruments in a nail salon, or alternatively, a method for providing services to customers of the nail salon. Various embodiments of such methods are discussed, infra.

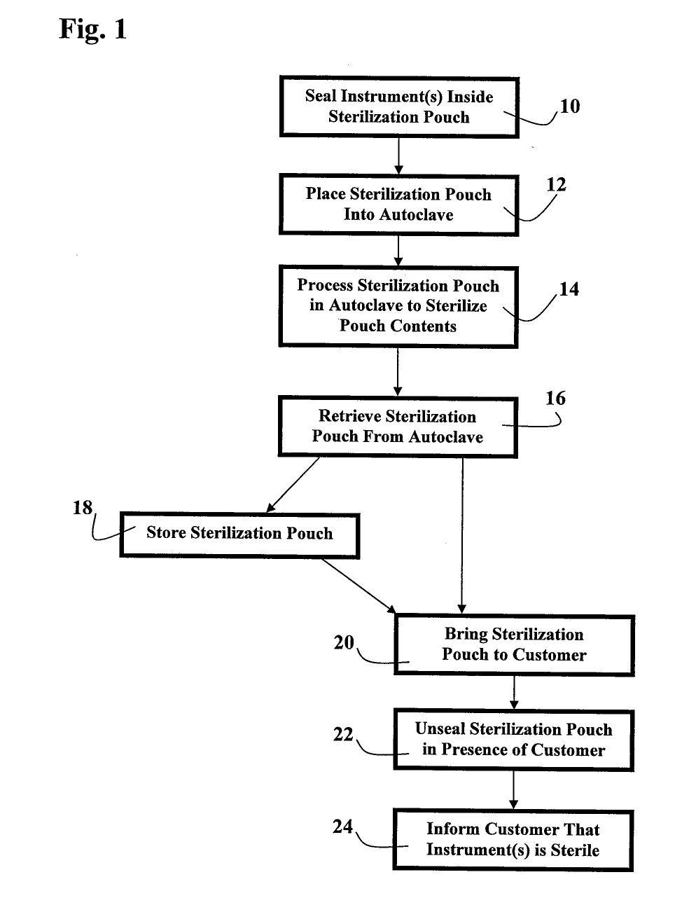

Referring to FIG. 1, a block diagram is presented, illustrating one method according to the present invention for providing services to a customer in a nail salon, where the customer is to receive a manicure and/or pedicure with a reusable instrument(s). The illustrated method may be used in conjunction with an instrument or instruments that have been used previously, or used for the first time. If the instrument(s) has been used previously, it is ideal that the instrument(s) be washed and rinsed prior to beginning the illustrated method. A preferred method of washing is by means of an ultrasonic bath. The illustrated method is primarily to be used in conjunction with reusable manicure/pedicure instruments, such as nail files, cuticle sticks, nail clippers and cuticle nippers.

According to FIG. 1, the instrument(s) is first sealed inside a sterilization pouch 10. Next, the sterilization pouch is placed into an autoclave 12. The autoclave then processes the pouch 14 thereby sterilizing and/or disinfecting the instrument(s) contained therein, after which the sealed pouch is retrieved from the autoclave 16. At this point, the sealed pouch may either be safely stored 18, or brought directly to the customer 20. Regardless of whether the sealed pouch is brought directly to the customer from the autoclave or alternatively, from storage, the next step of the process involves unsealing the pouch in the presence of the customer 22. The act of unsealing the pouch may by itself be sufficient to inform the customer that the contents of the pouch are sterile. However the method preferably includes explicitly informing the customer that the contents are sterile 24. This may be done in the form of a written legend on the package indicating that the contents of the sealed package are sterile, a verbal statement to that effect, and/or in a written document or sign not part of, or attached to the pouch. By so doing the customer gains a sense of ease, thereby increasing the likelihood that she/he will return for further manicures/pedicures. Thus, the subject invention offers the nail salon an extremely valuable incentive for its customers.

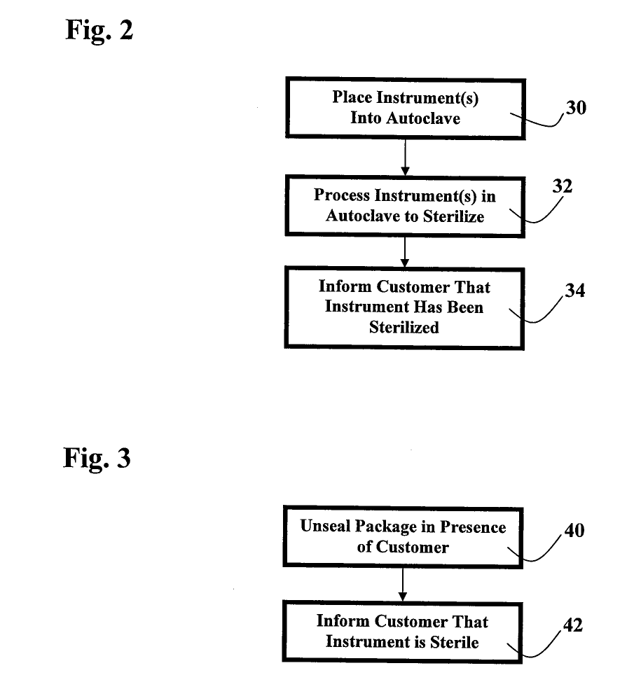

Referring now to FIG. 2, a method according to the present invention of sterilizing an instrument(s) to be used for a manicure and/or pedicure is illustrated. This method involves placing at least one of the instruments into an autoclave 30 and processing the instrument(s) 32 in order to sterilize it. The instrument may be placed in some type of container, but it is not essential to do so. Preferably, this method includes an additional step of somehow informing a customer that the instrument(s) has been sterilized 34. This may be achieved verbally or in writing, for example. Alternatively, once sterilization is complete, the instrument(s) may be placed into a bag or container, which, when opened in the presence of the customer, informs the customer that the instrument(s) has been sterilized.

Referring now to FIG. 3, a block diagram is presented, illustrating one method according to the present invention for providing services to a customer in a nail salon, where the customer is to receive a manicure and/or pedicure with a disposable instrument(s). This method involves unsealing a package containing at least one disposable instrument to be used for performing a manicure and/or pedicure on the customer. The unsealing of the package is performed in the presence of the customer 40. This act assures the customer that the disposable instrument has not been used on any other person. Preferably, this method includes a means to explicitly inform the customer that the instrument(s) is sterile 42. Such a means may be in the form of a written legend on the package indicating that the contents of the sealed package are sterile, a verbal statement to that effect and/or in a written document or sign not part of, or attached to the package.

In any of the embodiments of the present invention in which the pouch or package containing an instrument(s) is unsealed in the presence of a customer, the customer is assured that the instruments that will be used in the manicure and/or pedicure are sterile. Thus, the present invention: (1) protects the health of the customer; (2) provides the customer with confidence that instruments to be used on her/him are sterile; (3) is good for a nail salon's business because of the benefits enjoyed by its customers; and (4) protects a nail salon from potential liability.

While the invention has been described in detail and with reference to specific embodiments thereof, it will be apparent to one skilled in the art that various changes and modifications can be made therein without departing from the spirit and scope thereof.

What is claimed is:

1. A system for sterilizing and/or disinfecting instruments to be used for manicures and/or pedicures, said system comprising:
a. an autoclave; and
b. a sterilization pouch containing at least one of said instruments.

2. A method of sterilizing and/or disinfecting instruments to be used for manicures and/or pedicures, said method comprising the steps of:
a. placing at least one of said instruments into a sterilization pouch;
b. sealing said sterilization pouch; and
c. processing said sterilization pouch in an autoclave, thereby sterilizing and/or disinfecting of said instruments.

3. A method for providing services to a customer of a nail salon, said method comprising the steps of:
a. placing at least one instrument into a sterilization pouch, said instrument to be used in performing a manicure and/or pedicure on said customer;
b. sealing said sterilization pouch;
c. processing said sterilization pouch in an autoclave, thereby sterilizing said instrument; and
d. unsealing said sterilization pouch in the presence of said customer, prior to using said instrument to perform said manicure and/or pedicure on said customer.

4. The method of claim 3 further comprising the step of explicitly informing said customer that said instrument(s) is sterile.

5. The method of claim 4 wherein said step of explicitly informing said customer that said instrument(s) is sterile, is done verbally and/or by a writing.

6. A method for providing services to a customer of a nail salon, said method comprising the unsealing of a package containing at least one disposable instrument to be used for performing a manicure and/or pedicure on said customer, said unsealing of said package being performed in the presence of said customer.

7. The method of claim 6 further comprising the step of explicitly informing said customer that said instrument(s) is sterile.

8. The method of claim 7 wherein said step of explicitly informing said customer that said instrument(s) is sterile, is done verbally and/or by a writing.

9. A method of sterilizing and/or disinfecting instruments to be used for manicures and/or pedicures, said method comprising the steps of:
a. placing at least one of said instruments into an autoclave; and
b. processing said instrument(s) in said autoclave, thereby sterilizing said instrument.

10. The method of claim 9 further comprising the step of explicitly informing a customer on which said manicure and/or pedicure is to be performed, that said instrument(s) is sterile.
